(19) United States
(12) Patent Application Publication
(10) Pub. No.: US 2016/0228726 A1
(43) Pub. Date: Aug. 11, 2016

(54) APPLICATOR AND RECEPTACLE FOR A RADIOACTIVE SOURCE FOR BRACHYTHERAPY

(71) Applicant: IDTM GMBH, Recklinghausen (DE)

(72) Inventors: Michael FRIEBE, Recklinghausen (DE); Nassir NAVAB, München (DE)

(21) Appl. No.: 15/023,018

(22) PCT Filed: Sep. 18, 2014
(86) PCT No.: PCT/EP2014/069929

§ 371 Date: Mar. 18, 2016

(30) Foreign Application Priority Data
Sep. 19, 2013 (DE) 10 2013 110 375.8

(51) Int. Cl. A61N 5/10

(52) U.S. Cl. CPC A61N 5/1015; A61N 2005/1021

(57) ABSTRACT

Disclosed herein is an instrument kit and applicator for the localized internal irradiation of tumors or temporary post-operative cavities. For example, an applicator is disclosed in the form of a balloon for a radiation source of ionized radiation, which applicator enables an exactly dosed irradiation in the tissue concerned.

The invention resides on the field of medical device technology for the precisely localized internal irradiation of tumors or temporary post-operative cavities. The invention relates in particular to an applicator in the form of a balloon for a radiation source of ionized radiation, which applicator enables an exactly dosed irradiation in the tissue concerned.

The external irradiation of degenerated tissue for therapeutic purposes can in most cases not be limited solely to the respective target area since layers of healthy tissue situated between the tumor and the radiation source are inevitably penetrated by radiation. During this, healthy tissue is unnecessarily affected. In contrast to this, various approaches of the internal radiation therapy (brachytherapy) offer a possibility to restrict the undesired exposure to radiation of healthy tissue by reducing the distance between the radiation source and the target tissue. The therapeutic effect may likewise be optimized by the radiation source's precisely localized placement. Especially for the effective application of radiation having a reduced penetration depth such as for instance in the case of α-radiators such as uranium and thorium or of β-radiators such as for instance ⁹⁰Sr, ¹⁰⁶Ru, ^{186/188}Re, a direct contact of the receptacle with the target tissue may be desired for the purpose of a precisely adjustable radiation dose. In the practical application of the internal brachytherapy, however, this contact is made difficult by accumulating “wound secretion” so that precise dosing is impeded by unclear exposure conditions. In addition, such an implementation also allows surgical cavities to be irradiated which have formed due to tumor tissue having been surgically removed.

In light of the above, an applicator according to claim 1 and a kit according to claim 11 are proposed. The proposed applicator for internal brachytherapy acting as a radiation source overcomes the drawbacks described above of known devices. Further embodiments, modifications and improvements will result by means of the following description and the accompanying claims.

According to a first embodiment, an applicator for a radiation source for internal brachytherapy is proposed. The applicator comprises a tube with a tube wall having an inner side and an outer side. The tube exhibits a first, i.e. in the case of application anterior or patient-side opening, and a second or posterior opening facing away from the patient in the case of application, wherein a reversibly expandable membrane at least at the first opening is in connection with the tube in such a manner as to delimit an interior space enclosed by the tube wall from an exterior space, respectively an environment. The environment in particular represents a body cavity or a post-operative cavity to be subjected to brachytherapy.

Advantages will result from the targeted exposure of structures of the exterior space with the aid of a radionuclide disposed in the interior space or in the elastic membrane, with the radionuclide being isolated by the expandable membrane from the structure to be exposed. Since the expandable membrane is selected such with respect to its material composition and with respect to its thickness during the exposure that it is penetrated by or emits ionized radiation of the radionuclide, brachytherapy by means of the described applicator may be performed in a highly precise manner: The irradiation dose and the duration of exposure can be defined exactly. Known radionuclide receptacles for brachytherapy do not allow for any such precisely adjustable exposure duration.

According to a further embodiment, an applicator is proposed, in which the expandable membrane completely covers the tube's inner wall and closes the tube's anterior or first opening such that the expandable membrane, upon hydrostatic pressure at the posterior opening facing away from the patient being applied to the interior space enclosed by the tube, is expanded at the anterior, respectively patient-side opening in the form of a balloon. This causes the radiation source—being for instance in the form of a radioactive fluid—to be delimited from the environment by a wall of the balloon at the tube's anterior opening.

Advantages of this embodiment will mainly result from the surface of contact of the radionuclide for instance present within the balloon with structures outside the balloon being adjustable via a respective size of the balloon. As a whole, the size of the balloon's surface primarily determines the size of the structures within the exterior space treated by means of brachytherapy. Thus, the expansion of a treatable area in terms of surface area is adjustable by way of the balloon's size, respectively surface.

According to a further embodiment of the proposed applicator, the expandable and elastic membrane enclosing the radioactive fluid delimits the radioactive fluid with respect to the tube wall.

Hence, advantages arise for the applicator's multiple use with different radionuclides. A direct contact of the radioactive fluid with the tube is completely inhibited, for example.

According to a further embodiment, an applicator is proposed, in which the tube exhibits an outer diameter in the range of 0.5 to 30 mm, optionally of 2 to 10 mm, preferentially of 4 to 8 mm, and a wall thickness in the range of 0.02 to 1 mm, optionally of 0.05 to 0.25 mm.

Advantages arise from the comparatively small diameter of an opening for inserting the applicator, respectively the tube in a tissue structure to be subjected to brachytherapy.

According to a further embodiment, an applicator is proposed, in which the balloon's diameter in a circumferential direction reaches a value exceeding the tube's outer diameter twice to 200 times, optionally 3 to 100 times, preferentially 5 to 50 times.

Advantages of this embodiment result from the elastic membrane's high flexibility. During the expansion to its final size, it is able to adapt to the spatial dimensions of the structures in the exterior space such that an intimate contact of the radionuclide provided in the balloon, i.e. in the applicator's interior space, with the tissue to be subjected to brachytherapy can be established. It is to be noted in this context that the term “balloon” as used herein does not imply for the mass of the radionuclide provided from the tube's posterior end and enclosed outside the tube by the elastic membrane necessarily to have a spherical shape. Even with a typical balloon shape in the common linguistic use representing a rotation symmetrical shape, the balloon under the conditions prevailing here of a balloon entering into direct contact with a cavity in a tissue structure, preponderantly adopts the shape of the cavity, wherein the cavity's volume can be determined by means of diagnostic systems and thus also a precise radiation dosing is possible. The expandable membrane in a way lines a cavity in the exterior space and thus enables—via the elastic membrane's barrier—the contact with the inner side of the respective cavity with the radionuclide present in the possibly deformed balloon or at least in/on the wall thereof.

According to a further embodiment, an applicator is proposed, in which the expandable membrane is selected such that the balloon that can be formed at the anterior opening is reversible, so that in case of a hydrostatic pressure in the interior space of less than or equal to that in the exterior space, the tube's outer diameter determines an outer diameter of the applicator in the circumferential direction.

Particular advantages of this embodiment result from the fact that the balloon is completely collapsible, the applicator thus can be pulled back through the small opening previously used for inserting.

According to a further embodiment, an applicator is proposed furthermore comprising a pipe disposed in the interior space, yet in fluid isolation from the interior space which can be brought into direct fluid contact with the exterior space and permits a negative pressure to be generated in the exterior space when the tube wall's outer side at least at the posterior opening is fluid-sealed with respect to the exterior space. A direct mechanical contact between the membrane, for instance a balloon formed by the elastic membrane and the exterior space can thus be improved.

Advantages of this embodiment, for example, are that the balloon can thus be brought into particularly intimate contact with the inner surface of a cavity present in the outer space. Any fluids initially present within the exterior space can be sucked off and thus do not represent any additional barrier for the ionized radiation emitted from the radionuclide behind the elastic membrane.

According to a further embodiment, an applicator is proposed, in which said pipe which is partially disposed within the interior space is surrounded in the circumferential direction by the elastic membrane and on an outer side of the membrane forming the balloon merges in areas of the membrane which exhibit a different surface profile and/or a different roughness in mutually adjacent sections.

Advantages of this embodiment are for instance that not only the fluid accumulated directly at the pipe's anterior opening can be sucked off by way of negative pressure, but that structures even resting against the elastic membrane in the circumferential direction are drained. An intimate contact of the balloon's surface with the inner surface of the cavity present in the exterior space is thus achieved.

According to a further embodiment, an applicator is proposed, in which the tube wall differs on its inner side and its outer side in its material composition, and a layer extending from the outer side toward the inner side comprises a material which at least partially absorbs ionized radiation.

Advantages result from the fact that an effect of the ionized radiation emitted from the radionuclide is exclusively ensured on the exterior space's structures being in contact with the elastic membrane. There is no radiation impact on healthy tissue via the conduit used to insert the applicator.

According to a further embodiment, an applicator is proposed, in which a material which at least partially absorbs ionized radiation is disposed between the tube wall's inner side and outer side.

Advantages result from a simplified design of the tube wall. If an appropriate material of layer-like structure is utilized, a useful lifetime of the applicator can be maximized without impairment to the tube's surface quality.

According to a further embodiment, an instrument, respectively an instrument kit is proposed, comprising:—a tube (10) with a tube wall which has an inner side and an outer side, further comprising an anterior or first and a posterior or second opening which are interconnected by the tube wall, wherein the tube wall encloses an interior space;—an expandable membrane which is at least connected with the first or anterior opening of the tube so that the expandable membrane delimits the interior space with respect to the exterior space;—a pipe disposed in the interior space, yet in fluid isolation from the interior space, which pipe can be brought into direct fluid contact with the exterior space and permits a negative pressure to be generated in the exterior space when the tube is fluid-sealed with respect to the exterior space at least in some sections, preferably at least toward the second or posterior opening so that a contact area between the membrane and the exterior space is enlargeable.

Advantages of this kit result from the already described advantages of the device. The applicator may optionally be employed in an application-specific manner with arbitrary radionuclides in arbitrary formulation—for example with regard to a preferred dosing of the radionuclide in a carrier substance, with regard to a preferred viscosity of the formulation, with regard to a preferred temperature or a preferred volume of the balloon to be formed. Exemplary formulations of the radionuclide may be liquids, gases, gels, emulsions, dispersions, solutions or pastes. The described fluid sealing may be achieved when the exterior space rests or is caused to rest in the circumferential direction—for example at least by short-term mechanical pressing—against the tube's outer wall at least in some sections.

According to a further embodiment, a cross-sectional surface of the tube's first or anterior (in use patient-side) opening exhibits a commensurate or smaller cross-sectional area than in the direction toward the second opening in use facing away from the patient. This results in a conical shape of the tube which facilitates the inserting thereof into a body opening, for instance a wound cavity. In addition, an integrally conical shape of the tube enables a facilitated fluid sealing with respect to the body cavity, for instance a post-operative cavity.

The embodiments described above can be mutually combined in any arbitrary way. Several embodiments may be selected and mutually combined in this case. Likewise, all embodiments may be mutually combined while omitting single or several specific features.

According to the described embodiments, the emitter is typically embedded in the balloon. The applicator enables negative pressure to be generated at its front end, whereby wound openings are able to be closed on the one hand, and tissue fluids sucked off, on the other. The described embodiments enable the reduction of the impact on healthy tissue and the complete and precise dosing of the target tissue's radionuclide exposure, and thus overcome the drawbacks of known devices and methods described at the beginning.

The appended drawings illustrate embodiments of the proposed applicator and serve the purpose of explaining the principles of the invention in conjunction with the description. The elements of the drawings are represented relative to each other and not necessarily true to scale. Identical reference numerals correspondingly designate similar parts.

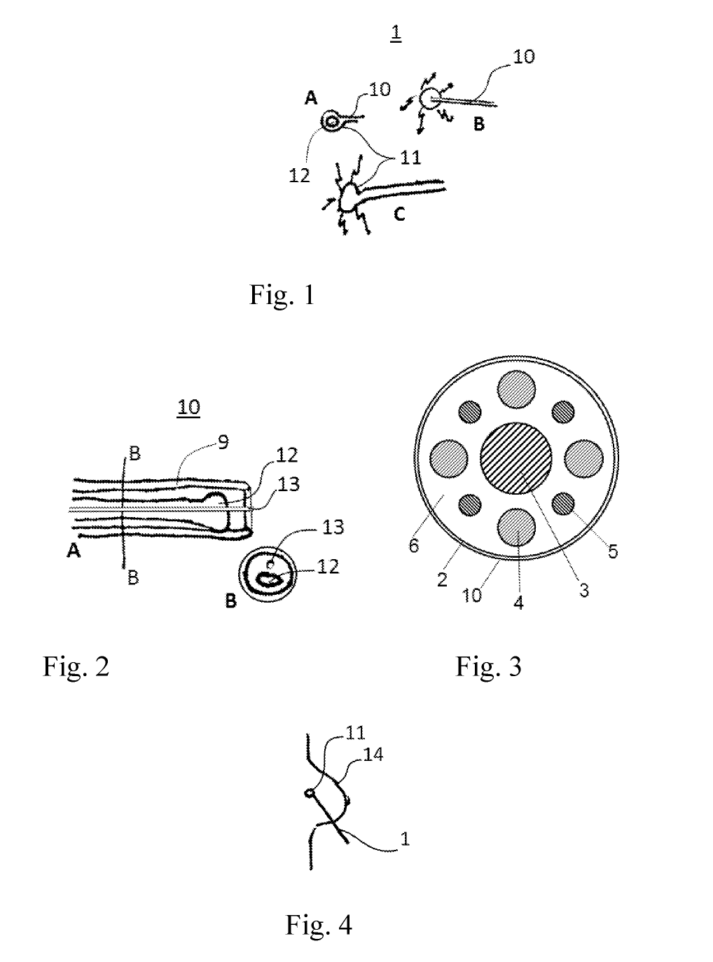

FIG. 1 shows an applicator for a radioactive radiation source for temporary brachytherapy;

FIG. 2 shows the structure of the applicator with the receptacle initially still contained therein according to one embodiment;

FIG. 3 shows an alternative embodiment of a cross-section of the applicator, comprising different conduits;

FIG. 4 shows an implementation of the applicator for brachytherapy in case of a mamma carcinoma.

Three different embodiments A, B and C of the applicator 1 in its respective configuration during brachytherapy are in particular outlined in FIG. 1. The applicator 1 typically comprises an elongate tube 10 and a balloon-like expansion disposed on the front end of the same. The balloon encloses a matrix 12 containing the radionuclide provided for the short-time irradiation, and thus prevents the radionuclide's direct contact with the environment. The matrix 12 may be for example in the form of a solution, an emulsion or a paste.

The balloon, respectively the balloon-like expansion is typically formed by a unilaterally closed hose 12 made of an elastomer. The balloon is not formed until the elastomer hose 12 is filled with the matrix. The balloon thus only forms when the elastomer hose expands unilaterally. Similar to a non-inflated air balloon, the elastomer hose is initially present within the tube 10 as a unilaterally closed capillary having an elastic wall. At the elastomer hose's posterior open end situated at the rear end of the tube 10 likewise not shown here, a matrix is pressed into the elastomer hose under pressure. This may be performed by a simple piston mechanism, for example a piston-operated pipette, a dosing device, for example a dosing pump, or using a syringe with a lockable piston.

Since the tube's 10 wall 9 is rigid, it will withstand the hydrostatic pressure of the matrix so that the elastomer membrane together with the matrix enclosed by it bulges out at the tube's front end and forms the balloon. Thus, an irradiation of the tissue enclosing the balloon may take place through the balloon's wall. As shown in FIG. 1B, the membrane forming the balloon may even be attached outside the tube 10, the charging of the balloon may thus be performed immediately via the tube. Likewise, as shown in FIG. 1C, the applicator may be realized in one piece. The material forming the balloon here has a greater material thickness in the area of the tube than at the front end thereof so that the balloon is formed at the tube's front end upon impingement of the matrix situated therein.

In order to improve the balloon's tissue contact with the surrounding tissue, an additional capillary 13 fluidically separated from the balloon may be used. An exemplary embodiment hereto is shown in FIG. 2. A capillary 13 extending inside the tube 10 serves the purpose of generating a negative pressure at the front of the applicator's 1 tube 10 already introduced into the tissue to be irradiated. For example, tissue fluid, respectively wound secretion may be pumped out via the capillary 13, while the balloon is being gradually bulged out by means of the matrix pressed in including the radionuclide 12. The balloon, respectively the unilaterally closed elastomer hose forming the balloon is realized to be separate according to the embodiment outlined here. FIG. 2A shows a longitudinal section of the front end of the tube 10. FIG. 2B shows a cross-section along the section plane designated B-B in FIG. 2A. According to preferred embodiments, the tube, at least at its anterior, patient-side end portion, exhibits an additional shielding wall 9. This shielding may be formed for example by a shielding means 2, for example a suitable absorber layer 2, at least embedded at the wall's 9 front-side end portion or applied to its inner side. As a suitable material, for example lead foil or an alloy containing lead, as well as tungsten or alloys containing tungsten or other absorber materials known to the skilled person are possible.

Said materials may be applied onto the inner side or the outer side or both sides of a metallic tube using various techniques known to the skilled person. Similarly, the layers applied—e.g. by means of CVD, a plasma treatment or by means of plating or sputtering techniques—subsequently may be provided with a passivation layer which prevents a direct contact of the absorber material with the environment.

Typically, such an absorber layer is appropriately surface-coated on both sides as well as on its front edge so as to prevent a contact with the environment. This presents the advantage of preventing corrosion of the absorber material, no contact of heavy metals with tissue or body fluids takes place, the tube 10 is protected from mechanical damage and can be subjected to the usual sterilization procedures.

According to a further embodiment, the capillary 13 may likewise extend inside the elastomer hose and exit at its anterior closed end. If the elastomer hose's outer surface exhibits additional structures at its front end, for example protuberances, grooves or corrugations, wound secretion may not only be collected and withdrawn directly from the anterior opening of the capillary 13 when applying negative pressure but also from the entire surroundings of the balloon being formed across the entire surface thereof. A particularly intimate tissue contact is achieved in this way. Such protuberances, grooves and small channels may be generated by adding suitable particles to the elastomer, but also by the only measure of providing an alternating wall thickness of different thickness and/or elasticity.

Advantageously, the proposed applicator in the form of the balloon charged with the radionuclide prevents the employed nuclide's direct tissue contact, on the one hand. Accordingly, an infiltration of the tissue by the nuclide is prevented. Due to the flexible and thin outer wall of the balloon-shaped receptacle, however, an outmost intimate contact with the tissue to be irradiated and thus an effective exposure during the brachytherapy are enabled. In addition, the tube 10 equipped with a shielding layer 2 prevents the undesired exposure of surrounding healthy tissue. On the other hand, the described constructional features facilitate substantially the receptacle's withdrawal upon expiry of the scheduled treatment time. As a whole, an exactly dosed irradiation can be achieved even in the event of cavities of complex shapes in the tissue concerned.

FIG. 3 illustrates a cross-section of the applicator according to an alternative embodiment. The tube 10 is here coated with a shielding means 2 on the inner side. The applicator's tube 10 may have various working channels. Apart from one continuous main working channel 3 for the elastomer hose forming a balloon, for example up to four additional continuous working channels 4 for tools or dosing measurements or for the additional diagnostic imaging as well as further up to 6 continuous working channels 5 for the localized generation of the desired vacuum may be provided.

FIG. 4 illustrates the implementation of the applicator 1 with a balloon 11 formed at the tube's first opening in the case of the brachytherapy of a mamma carcinoma of the female breast 14.

The applicator may be employed after surgical removal of a tumor. In this case, the wound opening remaining after the surgical intervention is closed by the negative pressure generated by means of the capillary 13 shown in FIG. 2 or by the channels 5 shown in FIG. 3.

According to further embodiments, the membrane forming the balloon may comprise an elastomer which itself is enriched with radionuclide by any suitable ways and means. To the skilled person, cross-linkable and chelating compounds are known for example which can be applied to the polymer membrane and cross-linked with the same or introduced into an elastic polymer membrane. Optionally, such molecules charged with radionuclide may be introduced directly into the elastic membrane or may serve the purposes of forming the very membrane. In such a case, the balloon is simply inflated with a radionuclide-free fluid. According to a modification of this embodiment, said fluid may in addition be tempered, for example cooled. This can be advantageous for a reduced blood circulation during the brachytherapy and a thus reduced damage of blood plasma cells.

Although specific embodiments have been presented and described here, it is within the scope of the present invention to modify the embodiments shown in an appropriate way without departing from the present invention's scope of protection. The following claims represent a first, non-binding attempt to define the invention in general.

1. An applicator for a radiation source for internal brachytherapy, comprising:
a tube, wherein the tube comprises:
a tube wall,
a first opening, and
a second opening; and
an expandable membrane:
enclosing a radioactive fluid and
being reversibly expandable while forming a balloon;
wherein the membrane is shaped such that the balloon is connected with the tube at least at its first opening, said first opening comprising an anterior or patient-side opening when the applicator is in use.

2. The applicator according to claim 1, wherein the expandable membrane is at least in some sections in mechanical contact with the tube wall of a front section of the tube so that, when hydrostatic pressure is applied to the radioactive fluid, the balloon can exit from the tube at the tube's first or patient-side opening.

3. The applicator according to claim 1, wherein the expandable membrane delimits the radioactive fluid from the tube wall.

4. The applicator according to claim 1, wherein the tube has at least in an area adjacent to the first opening an outer diameter of 0.5 to 30 mm, optionally of 2 to 10 mm, preferentially of 4 to 8 mm, and a wall thickness of 0.02 to 1 mm, optionally of 0.05 to 0.25 mm.

5. The applicator according to claim 1, wherein the balloon is expandable in at least one circumferential direction and exceeds the tube's outer diameter in an area adjacent to the first opening thereof twice to 200 times, optionally 3 to 100 times, typically 5 to 50 times.

6. The applicator according to claim 1, further comprising a pipe substantially disposed within the tube in fluid isolation from the tube and the balloon and having a first and a second opening being in direct fluid contact to an exterior space and allowing a negative pressure to be generated in the exterior space, when the tube is at least in some sections fluid-sealed with respect to the exterior space so that a contact area between the balloon's wall and the exterior space is enlargeable.

7. The applicator according to claim 6, wherein the pipe is surrounded by the expandable membrane in the circumferential direction and merges on an outer side of the membrane forming the balloon in mutually adjacent areas of the membrane which exhibit at least one of a different surface profile and a different roughness.

8. The applicator according to claim 1, wherein the tube wall on its inner side and its outer side differs in its material composition, wherein a layer extending from the outer side toward the inner side comprises a material which at least partially absorbs ionized radiation.

9. The applicator according to claim 1, wherein between the tube wall's inner side and outer side a material is disposed which at least partially absorbs ionized radiation.

10. An instrument kit for brachytherapy, comprising:
a tube with a tube wall, an inner side and an outer side of the tube wall, further comprising a first and a second opening which are interconnected by the tube wall, wherein the tube wall surrounds an interior space;
an expandable membrane enclosing a radioactive fluid and being arranged or shaped so as to be able to exit at the tube's first opening while forming a balloon when hydrostatic pressure is applied to the radioactive fluid;
a pipe substantially disposed in the interior area and being in fluid isolation from the interior area, which pipe can be brought into fluid contact with an exterior space outside the balloon and permits a negative pressure to be generated outside the balloon when the tube is fluid-sealed with respect to the exterior space at least in some sections so that a contact area between the membrane and the exterior space is enlargeable.

11. The instrument kit for brachytherapy according to claim 10, wherein a cross-sectional area at the first opening is equal to or smaller than a cross-sectional area in the direction toward or at the second opening.
